(19) United States
(12) Patent Application Publication
(10) Pub. No.: US 2006/0089557 A1
(43) Pub. Date: Apr. 27, 2006

(54) Method and apparatus to facilitate heart rate detection

(75) Inventors: Liliana Grajales, Bloomingdale, IL (US); Bryn A. Martin, Woodridge, IL (US); Ion V. Nicolaescu, Carpentersville, IL (US); Iwona Turlik, Barrington, IL (US)

Correspondence Address: FITCH EVEN TABIN AND FLANNERY, 120 SOUTH LA SALLE STREET, SUITE 1600, CHICAGO, IL, 60603-3406, US

(73) Assignee: Motorola, Inc.

(21) Appl. No.: 10/974,874

(22) Filed: Oct. 27, 2004

(51) Int. Cl. A61B 5/02

(52) U.S. Cl. 600/508; 600/473; 600/476

(57) ABSTRACT

A plurality of sensor clusters are provided (61) wherein at least some of the sensor clusters are comprised of at least one light sensitive sensor and a plurality of light emitters. These sensor clusters are disposed (62) in close proximity to a subject user and, in a preferred approach, at least some of the sensor clusters are disposed substantially distal to one another. The light emitters are caused (63) to emit light and the light sensitive sensors are used (64) to detect interactions as between the subject user and the emitted light. These detected interactions are then used (65) to determine the user's heart rate. In a preferred embodiment, the sensed interaction information is dynamically modified as a reflection of how valid the information trajectory appears to be at any given time.

### TECHNICAL FIELD

This invention relates generally to heart rate detection and more particularly to the use of light to detect a heart rate.

### BACKGROUND

An individual's heart rate (i.e., the periodicity of that individual's heartbeats, typically denoted as beats per minute) comprises an important indicator of that individual's present or near-term physical well being. Detection of the pulse of the heart permits calculation of a corresponding beat-to-beat heart rate. The beat-to-beat heart rate, in turn, permits calculation of heart rate variability for that individual. Heart rate variability facilitates an ability to assess the potential onset of coronary distress such as ventricular arrhythmia. This has potential important application in certain public service applications. For example, recent statistics indicate that approximately 50% of all firefighter's job-related deaths result from a coronary event, and approximately 25% of job-related deaths for law enforcement personnel such as police are due to a similar cause.

Unfortunately, a cost-effective, robust, and reliable mechanism to permit on-the-job monitoring of this sort for public safety personnel remains unmet. There are, of course, numerous available products that provide a measure of an individual's heartbeat. One example is a heart rate sensor chest strap that uses electrodes to detect the heart's electrical performance. Such a form factor comprises an unsatisfactory solution for many public safety personnel. This mechanism requires separate, and early, careful placement and installation, and hence its usage remains at odds with the time-critical nature of the public safety paradigm. It may be expected that, at least some of the time, an individual will decline to take the required time to don and/or test the apparatus for proper placement and functionality.

Another prior art solution comprises a watch that measure heart rate. This device, however, does not provide continuous monitoring. It must usually be momentarily activated by the individual through contact with a finger on an opposing hand. Such an approach does not serve well to provide sufficient and on-going data to permit calculation of heart rate variability.

Yet another approach utilizes a ring bearing an infrared sensor that can measure the wearer's pulse. In many instances, however, public safety personnel such as firefighters often use their hands in an aggressive manner (pulling, pushing, carrying, or otherwise wielding heavy objects, for example). Such actions present the likelihood of introducing signal artifacts that will distort a resultant heart rate variability calculation.

Other mechanisms and form factors exist as well, including devices that clip to the ear lobe. In general, however, such prior art approaches tend to require an inappropriate amount of time to properly don and locate, are subject to dislodgement during anticipated use, and/or are unduly sensitive to the harsh operating conditions of a public safety worker and either readily fail to adequately track the beat of the heart or introduce sufficient noise to render their usage problematic when seeking to detect heart rate variability.

### BRIEF DESCRIPTION OF THE DRAWINGS

The above needs are at least partially met through provision of the method and apparatus to facilitate heart rate detection described in the following detailed description, particularly when studied in conjunction with the drawings, wherein:

FIG. 1 comprises a comprises a block diagram as configured in accordance with various embodiments of the invention;

FIG. 2 comprises a schematic view as configured in accordance with various embodiments of the invention;

FIG. 3 comprises a schematic view as configured in accordance with various embodiments of the invention;

FIG. 4 comprises a schematic view as configured in accordance with various embodiments of the invention;

FIG. 5 comprises a front elevational schematic view as configured in accordance with various embodiments of the invention;

FIG. 6 comprises a flow diagram as configured in accordance with various embodiments of the invention;

FIG. 7 comprises a block diagram as configured in accordance with various embodiments of the invention; and

FIG. 8 comprises a graph that illustrates two illustrative exemplary sensor signals in accordance with various embodiments of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will also be understood that the terms and expressions used herein have the ordinary meaning as is usually accorded to such terms and expressions by those skilled in the corresponding respective areas of inquiry and study except where other specific meanings have otherwise been set forth herein.

### DETAILED DESCRIPTION

Generally speaking, pursuant to these various embodiments, an apparatus to facilitate heart rate detection comprises at least one sensor cluster that comprises at least one light sensitive sensor and a plurality of light emitters that are disposed closely proximal to the light sensitive sensor and a heart rate detector that operably couples to the sensor cluster. In a preferred approach, a plurality of such sensor clusters are disposed in close proximity to a subject user. The light emitters are caused to each emit light and the light sensitive sensors detect interactions between the subject user and the emitted light. Those interactions are then used to determine a heart rate for the subject user.

Depending upon the needs of a given application, the light emitters can all emit light having a substantially same wavelength, or at least one or more of the light emitters can emit light having a different wavelength. Such emitted light can comprise, for example, an infrared wavelength, a visible light wavelength, and so forth. Pursuant to one approach, the light emitters and light sensitive sensor can be positioned as a grouped cluster. Pursuant to another approach such elements are disposed closely proximal to one another in a substantially co-linear orientation.

Pursuant to a preferred approach, when a plurality of sensor clusters are deployed with respect to a single individual, a determination can be made regarding at least one signal quality factor (such as, but not limited to, apparent noise content and/or apparent signal accuracy) for at least some of the detection interactions. This determination can be used to provide a corresponding alteration value. This alteration value, in turn, can be used to modify at least one of the interactions prior to using the interactions to determine the subject user's heart rate. For example, this alteration value can comprise a gain value that modifies such an interaction through application of the gain value to the interaction.

By so altering a value as corresponds to at least one such interaction as a function, at least in part, of likely validity of the value, and then using the altered value when using the interactions to determine a subject user's heart rate, more robust performance can be expected. This, in turn, permits greater latitude with respect to the form factor and interface requirements of a useful heart rate detection system.

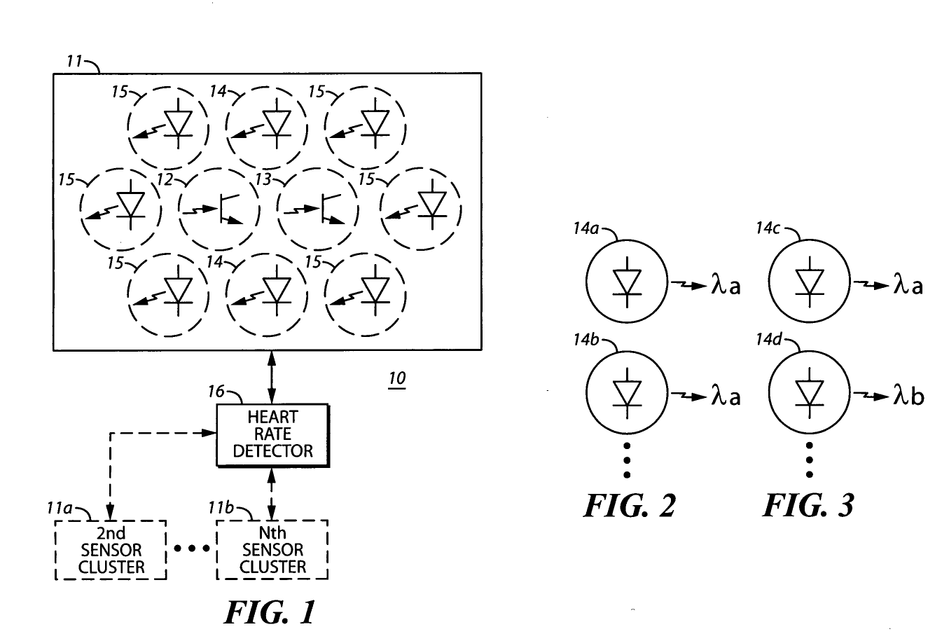

These and other benefits may become more evident upon making a thorough review and study of the following detailed description. Referring now to the drawings, and in particular to FIG. 1, an illustrative embodiment of an apparatus 10 to facilitate heart rate detection comprises a sensor cluster 11. This sensor cluster 11 comprises at least one light sensitive sensor 12 and may optionally include additional light sensitive sensors 13 as desired and/or as may be appropriate to meet the needs of a given application. The sensor cluster 11 further comprises a plurality of light emitters 14 that are disposed closely proximal to the light sensitive sensor 11 (or sensors). These light sensitive sensors and light emitters can be any presently known or hereafter-developed components as may effectively meet the needs of a specific context. For example, sensors/emitters that operate using infrared wavelength or visible light wavelengths may be suitably employed for these purposes.

Pursuant to one approach, and referring momentarily to FIG. 2, the light emitters 14 may all emit light having an identical or substantially similar wavelength. In such an embodiment, the light sensitive sensor will preferably be sensitive at least to light having that particular wavelength. Pursuant to another approach, and referring momentarily to FIG. 3, at least some of the light emitters 14 may emit light having different wavelengths. In such an embodiment, it will typically be appropriate to provide at least one light sensitive sensor for each such different wavelength.

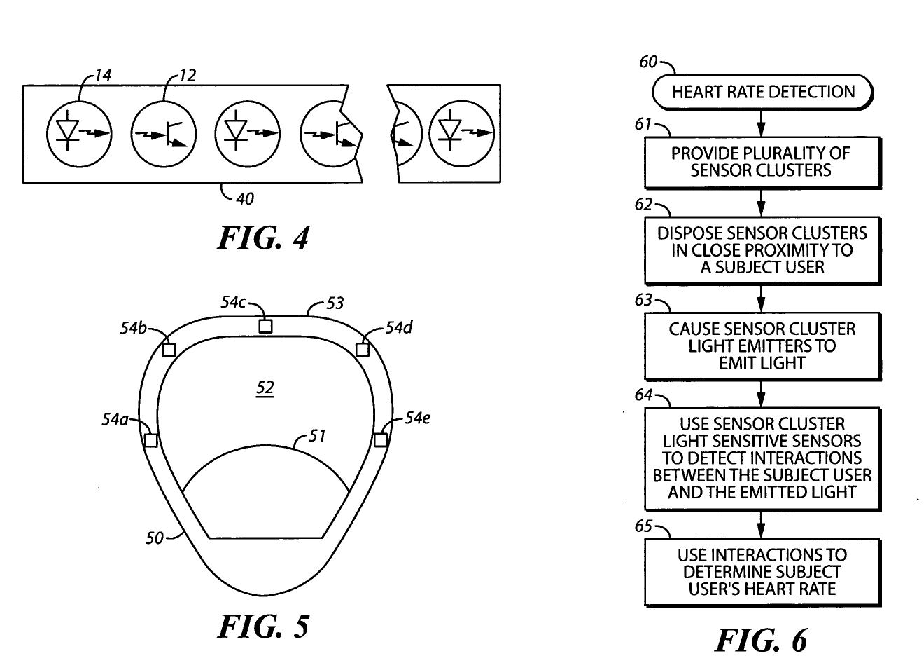

This sensor cluster 11 can be configured in a wide variety of form factors. As illustrated, the individual sensors and emitters can be closely clustered proximal to one another and share a common housing or other support platform. If desired, and referring momentarily to FIG. 4, at least some of the sensors 12 and emitters 14 can be disposed closely proximal to one another in a substantially co-linear orientation (as may be useful, for example, when the carrier 40 comprises a headband, wristband, or the like). In general, the apparatus 10 will comprise, at least in part, a wearable item and preferably a wearable item that is supported by a user's skin (to thereby facilitate an appropriate nexus between the active elements of the sensor cluster 11 and the user's skin). Considerably latitude exists, however, with respect to where the sensor cluster 11 portion of the apparatus 10 can be worn. For example, the wearable item portion of the apparatus 10 can be supported by any of a user's head, arm, leg, wrist, waist, or chest, to name a few.

Such light sensitive sensors 12 and light emitters 14 are generally well understood in the art. Further, these teachings are not particularly sensitive to selection of a particular sensor or emitter and are generally applicable to many such elements. Therefore, for the sake of brevity and the preservation of focus additional elaboration will not be provided here regarding such devices.

Referring again to FIG. 1, the sensor cluster 11 operably couples to a heart rate detector 16. This heart rate detector 16 can be integral to the sensor cluster 11 (such that the heart rate detector 16 is worn in a similar fashion to the sensor cluster 11) or can be physically distinct. In the case of the latter, the heart rate detector 16 can couple to the sensor cluster 11 via any appropriate link including both wired and wireless links as are well understood in the art.

If desired, and as may be preferred for many applications, the heart rate detector 16 can further operably couple to one or more additional sensor clusters 11a and 11b. Use of additional sensor clusters can provide useful information to permit more reliable detection of the subject user's heart rate as will be described more fully below. In general, such additional sensor clusters can be similar or identical to the sensor cluster 11 described above and can each include one or more light sensitive sensor and a corresponding plurality of light emitters that are again disposed closely proximal to the light sensitive sensor. When using additional sensor clusters, it is also possible to have the light emitters of, for example, a second sensor cluster 11a use a light wavelength that is different than the light emitters of the first sensor cluster 11. Other combinations are of course possible if desired. For example, a first one of the sensor cluster could comprise light emitters that all use a common light wavelength and another of the sensor clusters could comprise light emitters that use a mix of light wavelengths.

In many cases, when using a plurality of sensor clusters, it will be desirable to dispose at least some of the sensor clusters substantially distal to one another. For example, and referring now to FIG. 5, when a plurality of such sensor clusters 54a-54e are disposed within a facemask 50, it can be useful to disperse the sensor clusters 54a-54e around a frame 53 that surrounds a transparent faceplate member 52 and that encapsulates a breathing nosepiece 51. In particular, it can be helpful to dispose these sensor clusters in a spaced relationship to one another such that the sensor clusters are each effectively monitoring a substantially different portion of the user's skin and the light sensitive sensor(s) of each sensor cluster are primarily responding to light as emitted by the light emitters of their own corresponding sensor cluster.

So configured, light emitted from the light emitters impinges upon the skin of the wearer. This light interacts with the user in a known manner that varies in a relatively predictable manner as a function of the wearer's pulse rate. That is, the momentary increase of blood pressure due to a heartbeat will cause a variation in the reflected light as sensed by a corresponding light sensitive sensor. Use of light for the general purpose of detecting a heartbeat comprises a known area of endeavor. The above-described embodiments, however, offer particular advantages in this regard when deployed in more difficult monitoring situations such as those mentioned above. In particular, the use of multiple light emitters as described can aid in assuring a usable signal notwithstanding a physically noisy and highly dynamic operating environment.

Such an apparatus, or any other enabling apparatus as may be available in a given instance, can be employed to effect a heart rate detection process 60 such as that illustrated in FIG. 6. Pursuant to this process 60 one provides 61 a plurality of sensor clusters wherein each of the sensor clusters comprise at least a single light sensitive sensor and a plurality of light emitters that are disposed closely proximal to the light sensitive sensor. These sensor clusters are then disposed 62 in close proximity to a subject user and preferably in a distal relationship to one another. In particular, these sensor clusters are disposed to be in near or close contact with the user's skin to thereby more readily facilitate light-based heart rate detection.

So deployed, this process 60 then causes 63 the plurality of light emitters that comprise the sensor clusters to each emit light. If desired, such light emission can be continuous or substantially continuous. Since power consumption will often comprise a design concern, however, it will usually be preferable to effect this process using a reduced duty cycle. For example, useful results can be expected using a duty frequency of 10% when employing a light frequency of 400 Hz. (The fastest adult human heart typically will not exceed 4 Hz, so a pulse signal sampled at 400 Hz will provide 100 data points per beat under even these relatively extreme circumstances.)

This process 60 then uses 64 the light sensitive sensors to detect the expected interactions between the subject user and the light as emitted by the plurality of light emitters. These interactions are then used 65, in turn, to determine a corresponding heart rate for the subject user. The heart rate can then be used to calculate heart rate variability for the subject user as noted above if desired. In a preferred embodiment, and as will be described in more detail below, when a plurality of sensor clusters are deployed, this step preferably comprises processing the interaction information from each of the sensor clusters in combination with each other in order to determine the subject user's heart rate.

One advantage of deploying multiple sensor clusters in a distal orientation with respect to one another is that each sensor cluster will experience a different local physical environment. For example, when deployed within a face mask for breathing equipment, some of the sensor clusters may have a relatively optimum placement with respect to the wearer's skin while other sensor clusters may be less optimally placed at any given moment depending upon, for example, movement of the wearer, contact between the face mask and other objects, and so forth. In many cases, momentary dislocation of one or more of the sensor clusters will nevertheless not unduly adversely impact one of more of the remaining sensor clusters.

Using 65 the detected interactions to determine a user's heart rate can therefore beneficially further comprise making a determination regarding at least one signal quality factor (such as but not limited to noise content and/or apparent signal accuracy) for at least some (and preferably all) of the interactions to provide at least one alteration value and use of that alteration value to modify at least one of the interactions prior to using the interactions to determine the user's heart rate. For example, and as will be described below in more detail, such dynamic modifications can be employed to alter a gain value that is, in turn, applied to a signal that corresponds to the interaction information to thereby facilitate minimizing reliance upon interaction information that appears suspect while fostering reliance upon interaction information that appears to be more reliable.

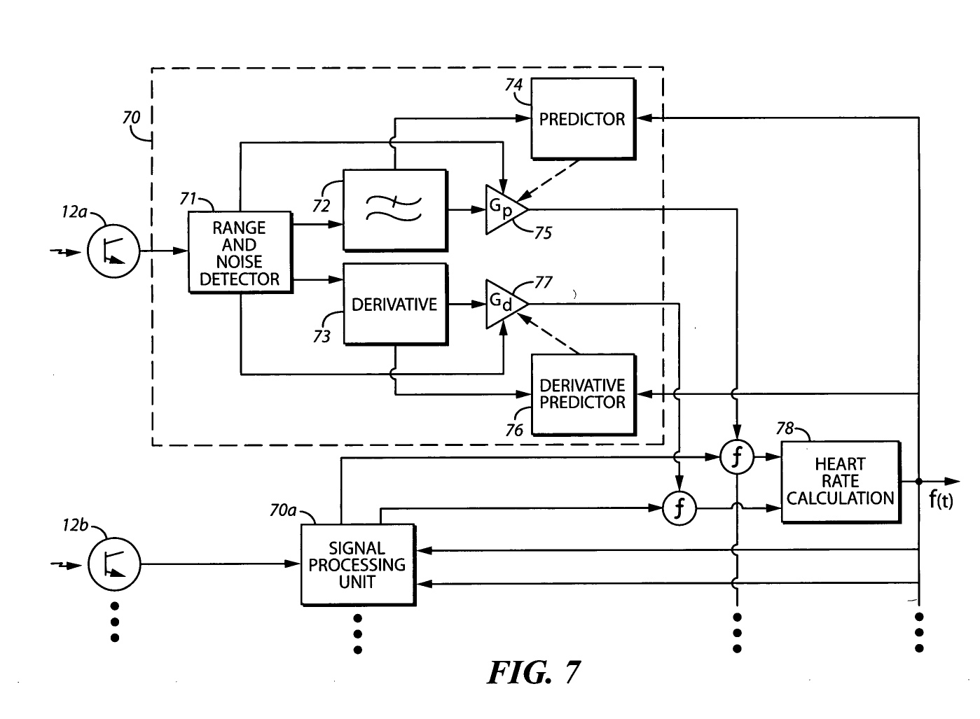

Referring now to FIG. 7, an apparatus to support and illustrate such an approach will be described. In this description, a signal processing unit 70 for a single light sensitive sensor 12a will be described, it being understood that additional such light sensitive sensors (represented by light sensitive sensor 12b) and their corresponding signal processing units 70a are provided as appropriate.

The signal processing unit 70 provides a block 71 comprising a frequency and magnitude range and noise level detector and pre-filter that receives the light sensitive sensor 12a output. This frequency and magnitude range and noise level detector and pre-filter 71 provides four outputs with a first output coupling to a first amplifier 75 having a variable gain Gₚ. This variable gain Gₚ is increased or decreased depending on the detected frequency and magnitude range and noise level. A second output is coupled to a second amplifier 77 having a variable gain G_{d}. This variable gain G_{d} is reduced or increased by the range and noise level detection of the block 71. The third output signal from this block 71 is a pre-filtered signal 12a directed to a low pass filter system 72 with cut off frequencies, in this embodiment, of 40 Hz and below. The fourth output signal from this block 71 is the pre-filtered signal 12a coupled to a derivative processor 73 that extracts the derivative of the incoming signal. The low pass filter 72 and the derivative processor 73 then each provide an output to a corresponding one of the first and second amplifier 75 and 77 wherein the first amplifier 75 has a variable gain Gₚ and the second amplifier 77 has a variable gain G_{d}. Each amplifier's 75 and 77 variable gain value is continuously influenced by the two outputs of this block 71.

In a preferred approach, these gain values are also influenced by corresponding prediction calculations. In the case of the amplifier 75 that couples to the low pass filter 72, the low pass filter 72 further couples to a predictor 74 that processes that incoming data in comparison with respect to a current overall heart rate calculation as per the function f(t) from a heart rate calculation unit 78. The resultant signal represents a sense of how well, or how poorly, the current trajectory of the incoming low pass signal corresponds to a current trajectory of a best overall calculation of the heart rate. This, in turn, is used to vary the gain Gₚ of the amplifier 75 for the low pass filter 72. So configured, when the behavior of the low pass filtered version of the signal from this particular light sensitive sensor 12a appears to correspond well with an overall view of the subject's heart beat, that low pass filtered version of the signal can be emphasized or at least not de-emphasized with respect to its subsequent use during calculation of the heart rate. Similarly, when the low pass filtered version of the signal from this light sensitive sensor 12a appears suspect (due to noise or any other cause or reason), the low pass filtered version of the signal can be de-emphasized accordingly and hence ameliorate the influence of that suspect signal with respect to calculation of the subject user's heart rate.

A similar process occurs for the derivative processed signal path. A derivative predictor 76 processes incoming information from the derivative processor 73 and compares that result against a similarly processed version of the overall heart rate calculation. Again, that comparison serves to provide information that is used to influence the gain G_{d} and thereby influence the extent to which this calculation impacts the heart rate calculation.

The gain modified outputs of the low pass filter 72 and the derivative processor 73 are then linearly combined, respectively, with the corresponding signals from other available light sensitive sensors 12b with the resultant combined signals then informing a standard heart rate calculation process 78 to yield a present determination f(t) regarding the subject's heart rate.

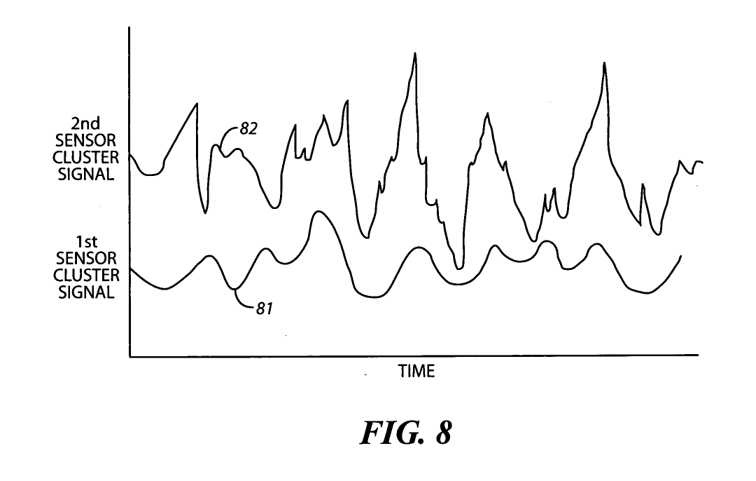

So configured, a plurality of sensor clusters can be disposed in various ways and/or in various monitoring locations with respect to a wearer's skin. By providing processing that aids in dynamically minimizing the contribution from sensor clusters that are presently providing unreliable information, one can also avoid, to a significant extent, a need for specialized placement and/or attention to the sensor clusters during use. In effect, to a large degree, a user can simply don their usual work apparel and/or pay only brief attention to installation of the sensor cluster(s) carrier item. Upon installation and/or during use, some of the sensor clusters may well be non-optimally placed, but other sensor clusters are more likely to be sufficiently well situated to permit accurate heart rate detection. To illustrate, and referring now to FIG. 8, a first sensor cluster signal 81 may be providing a relatively accurate portrayal of the subject's heart rate while a second sensor cluster signal 82 may be exhibiting considerable noise. A process such as that described above accommodates this kind of mildly chaotic and unpredictable paradigm while also tending to ensure the provision of accurate and useful information regarding the user's heart beat by minimizing the contribution of a noisy signal while maintaining or enhancing the relative contribution of a seemingly accurate signal.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept.

We claim:

1. An apparatus to facilitate heart rate detection, comprising:
a sensor cluster comprising:
a light sensitive sensor;
a plurality of light emitters disposed closely proximal to the light sensitive sensor;
a heart rate detector operably coupled to the sensor cluster.

2. The apparatus of claim 1 wherein the plurality of light emitters each emits light having a substantially same wavelength.

3. The apparatus of claim 1 wherein the substantially same wavelength comprises one of:
an infrared wavelength;
a visible light wavelength

4. The apparatus of claim 1 wherein the sensor cluster comprises a plurality of light sensitive sensors.

5. The apparatus of claim 1 wherein the sensor cluster comprises only a single light sensitive sensor.

6. The apparatus of claim 1 and further comprising:
a second sensor cluster comprising:
a second light sensitive sensor;
a second plurality of light emitters disposed closely proximal to the second light sensitive sensor;
and wherein the heart rate detector is further operably coupled to the second sensor cluster.

7. The apparatus of claim 6 wherein the second sensor cluster is disposed substantially distal to the first sensor cluster.

8. The apparatus of claim 6 wherein:
the plurality of light emitters as correspond to the sensor cluster each emits light having a substantially same wavelength;
the plurality of light emitters as correspond to the second sensor cluster comprise at least two light emitters that each emit light having a substantially different wavelength as compared to one another.

9. The apparatus of claim 1 wherein the apparatus comprises a wearable item.

10. The apparatus of claim 9 wherein the wearable item comprises an item that is supported by a user's skin.

11. The apparatus of claim 10 wherein the wearable item is supported by at least one of a user's:
head;
arm;
leg;
wrist;
waist;
chest.

12. A method to facilitate heart rate detection, comprising:
providing at least two sensor clusters, each comprising:
a light sensitive sensor;
a plurality of light emitters disposed closely proximal to the light sensitive sensor;
disposing the at least two sensor clusters in close proximity to a subject user;
causing the plurality of light emitters to each emit light;
using the light sensitive sensors to detect interactions between the subject user and the light as emitted by the plurality of light emitters;
using the interactions to determine a heart rate for the subject user.

13. The method of claim 12 wherein the interactions detected by the light sensitive sensors are processed in combination with each other in order to determine the heart rate for the subject user.

14. The method of claim 12 and further comprising:
disposing a second sensor cluster of the at least two sensor clusters in close proximity to a subject user but distal to a first sensor cluster of the at least two sensor clusters;
causing the plurality of light emitters for the second sensor cluster to each emit light;
using the light sensitive sensor for the second sensor cluster to detect another interaction between the subject user and the light as emitted by the plurality of light emitters for the second sensor cluster;
using the another interaction in combination with the interaction to determine a heart rate for the subject user.

15. The method of claim 12 wherein providing at least two sensor clusters that each comprise a light sensitive sensor further comprises providing at least one sensor cluster comprising a plurality of light sensitive sensors.

16. The method of claim 15 wherein providing at least two sensor clusters that each comprise a plurality of light sensitive sensors further comprises providing at least one sensor cluster comprising a plurality of light sensitive sensors and a plurality of light emitters, wherein the plurality of light sensitive sensors and the plurality of light emitters are disposed closely proximal to one another.

17. The method of claim 16 wherein the plurality of light sensitive sensors and the plurality of light emitters are disposed closely proximal to one another in a substantially co-linear orientation.

18. The method of claim 12 wherein using the interactions to determine a heart rate for the subject user further comprises:
making a determination regarding at least one signal quality factor for at least some of the interactions to provide at least one alteration value;
using the at least one alteration value to modify at least one of the interactions prior to using the interactions to determine the heart rate for the subject user.

19. The method of claim 18 wherein the at least one signal quality factor comprises at least one of:
noise content;
apparent signal accuracy.

20. The method of claim 18 wherein the at least one alteration value comprises a gain value.

21. The method of claim 20 wherein using the at least one alteration value to modify at least one of the interactions prior to using the interactions to determine the heart rate for the subject user further comprises:
applying the gain value to at least one of the interactions prior to using the interactions to determine the heart rate for the subject user.

22. The method of claim 12 wherein using the interactions to determine a heart rate for the subject user further comprises:
altering a value as corresponds to at least one of the interactions as a function, at least in part, of likely validity of the value to provide an altered value;
using the altered value when using the interactions to determine the heart rate for the subject user.
